(19) United States
(12) Patent Application Publication
(10) Pub. No.: US 2017/0296567 A1
(43) Pub. Date: Oct. 19, 2017

(54) Decubitus Ulcer Salve

(71) Applicants: Tammy L. Romero, Bartlett, TN (US); Stephen L. Gipson, Memphis, TN (US); Van Jones, Memphis, TN (US)

(72) Inventors: Tammy L. Romero, Bartlett, TN (US); Stephen L. Gipson, Memphis, TN (US); Van Jones, Memphis, TN (US)

(21) Appl. No.: 15/205,413

(22) Filed: Jul. 8, 2016

Related U.S. Application Data
(60) Provisional application No. 62/322,021, filed on Apr. 13, 2016.

(51) Int. Cl. A61K 31/7036; A61K 45/06; A61K 31/65; A61K 31/635; A61K 31/573; A61K 31/4174; A61K 31/203; A61K 31/10; A61K 9/06; A61K 47/10; A61K 9/00

(52) U.S. Cl. CPC A61K 31/7036; A61K 47/10; A61K 45/06; A61K 31/65; A61K 31/635; A61K 9/0014; A61K 31/4174; A61K 31/203; A61K 31/10; A61K 9/06; A61K 31/573

(57) ABSTRACT

A medical salve designed to address and treat bedsore, also known as decubitus ulcers. The medical salve includes a plurality of ingredients that each has their own unique beneficial properties. The plurality of ingredients includes a quantity of antibiotic powder composition, a quantity of antifungal powder, a quantity of topical steroid powder, a quantity of ethoxydiglycol, a quantity of silver sulfadiazine cream, a quantity of retinoic acid powder, a quantity of dimethyl sulfoxide solution, and a quantity of oleo plasticized base. The antibiotic powder composition, the antifungal powder, the topical steroid powder, the dimethyl sulfoxide solution, and the retinoic acid are the main active ingredients in the medical salve that are designed to address the symptoms of bedsores as well as expedite the healing process. The silver sulfadiazine cream, the ethoxydiglycol, and the oleo plasticized base are used as the base materials for medical salve.

The current application claims a priority to the U.S. Provisional Patent application Ser. No. 62/322,021 filed on Apr. 13, 2016.

### FIELD OF THE INVENTION

The present invention relates generally to medications designed to treat skin disorders. More specifically, the present invention is a salve composition designed to address and treat bedsores, also known as decubitus ulcers.

### BACKGROUND OF THE INVENTION

Bedsores, also known as pressure ulcers or decubitus ulcers, are injuries to the skin resulting from prolonged pressure being applied on the skin. Areas that are particularly susceptible to bedsores include regions of the body that are over a bony prominence, i.e. regions of the body where bone is relatively close to the skin surface such as the heel, ankle, hips, and tailbone. When external pressure is applied to said regions blood flow is partially or completely obstructed to the covered tissue. The lack of blood flow causes lesions and necrosis in the affected region. People that are at most risk to develop bedsores are those with pre-existing medical conditions that limit movement and as a result are bedridden or confined to a wheelchair. If left untreated, bedsores may lead to sepsis, cellulitis, bone and joint infections, or even cancer.

The present invention serves to treat bedsores, diabetic ulcers, and aids in healing wounds in animals. The present invention aids in the healing of such wounds and shows results in about five days. The present invention may be used alone or in conjunction with intravenous therapy, oral antibiotics, medicated bandages or wound care gels/ointment/creams. The present invention comprises combination of gentamycin powder, miconazole powder, hydrocortisone powder, neomycin powder, doxycycline powder, ethoxydiglycol, silver sulfadiazine cream, retinoic acid powder, dimethyl sulfoxide solution, and an oleo plasticized base.

### BRIEF DESCRIPTION OF THE DRAWINGS

FIG. 1 is block diagram depicting the ingredients of the present invention.

FIG. 2 is block diagram depicting the ratio of the ingredients within the present invention.

FIG. 3 is a block diagram depicting the constituents of the antibiotic powder composition.

FIG. 4 is a block diagram depicting the constituents of the analgesic powder.

FIG. 5 is a block diagram depicting the constituents of the topical steroid.

### DETAIL DESCRIPTIONS OF THE INVENTION

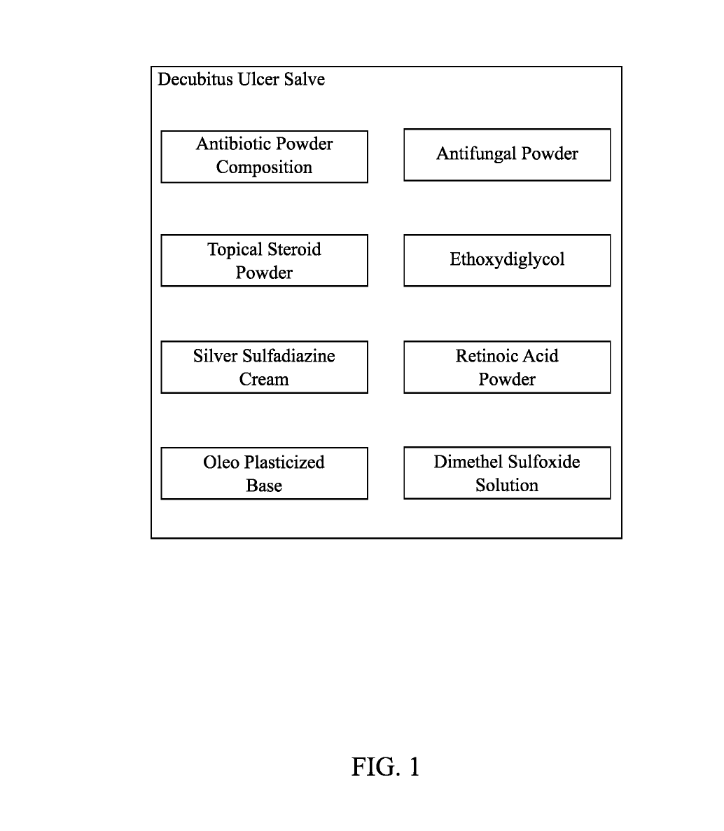

All illustrations of the drawings are for the purpose of describing selected versions of the present invention and are not intended to limit the scope of the present invention. The present invention is a decubitus ulcer salve that is used as a topical treatment to treat bedsores as well as address the associated symptoms. As can be seen in FIG. 1, the present invention mainly comprises a quantity of antibiotic powder composition, a quantity of antifungal powder, a quantity of topical steroid powder, a quantity of ethoxydiglycol, a quantity of silver sulfadiazine cream, a quantity of retinoic acid powder, a quantity of dimethyl sulfoxide solution, and a quantity of oleo plasticized base. Each of the ingredients is included to specifically address various symptoms of bedsores, thus expediting the healing process for said bedsores. Traditionally, bedsore medications are in the form of a salve, an ointment or cream. In relation to the present invention the quantity of antibiotic powder composition, the quantity of antifungal powder, the quantity of topical steroid powder, the quantity of ethoxydiglycol, the quantity of silver sulfadiazine cream, the quantity of retinoic acid powder, the quantity of dimethyl sulfoxide solution, and the quantity of oleo plasticized base are heterogeneously combined into a topical mixture. The topical mixture allows for quick and easy application of the present invention.

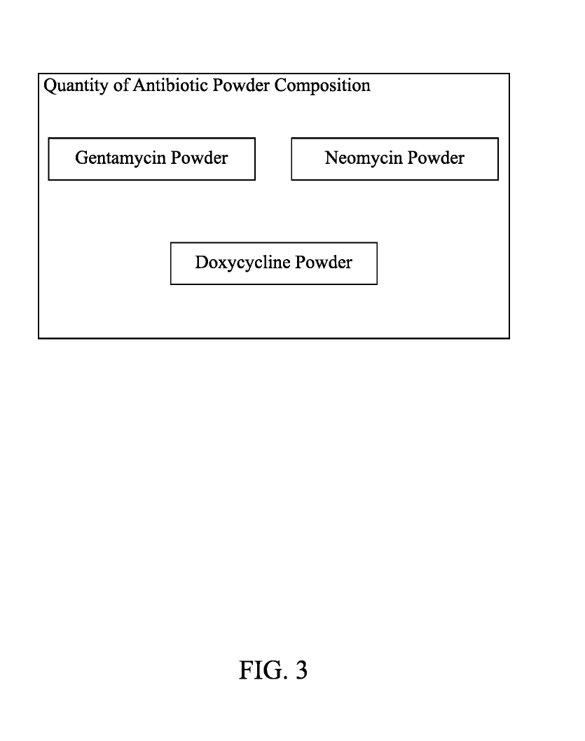

One of the main factors that slows down the healing process for bedsores is bacterial infection as the wounds can easily become breading grounds for various bacteria. One effect of bacteria in and around the wound is the buildup of biofilm, a complex structure comprising colonies of bacteria which adhere to the adjacent surfaces and stall the healing processes by keeping the wound inflamed. The quantity of antibiotic powder composition prevents infections from growing in and around the wound as well as aids in the break-down of biofilm, thus speeding up the healing processes. The quantity of antibiotic powder composition comprises a plurality of antibiotics in order to address a broad spectrum of bacteria. More specifically, referring to FIG. 3, the quantity of antibiotic powder composition is selected from a group of: a quantity of gentamycin powder, a quantity of neomycin powder, a quantity of doxycycline powder, and any combination thereof. Each of the antibiotics is used to treat and/or prevent the growth of a specific set of bacteria. In the preferred embodiment of the present invention, the quantity of antibiotic powder composition is about 5.25%, by weight, of the topical mixture.

Gentamycin is a broad-spectrum antibiotic that is generally used to treat a variety bacterial infections such as bone infections, endocarditis, pelvic inflammatory disease, meningitis, pneumonia, urinary tract infections, and sepsis. Additionally, gentamycin is used to treat superficial skin infections, thus ideal for treating bedsores as one stage of bedsores includes the formation of blisters or shallow craters in the skin. In particular, gentamycin is active against gram-negative bacteria including Pseudomonas, Proteus, Escherichia coli, Klebsiella pneumoniae, Enterobacter aerogenes, Serratia, and the Gram-positive Staphylococcus. In the preferred embodiment, the quantity of gentamycin powder is about 2%, by weight, of the topical mixture.

Neomycin is an aminoglycoside antibiotic that is most often times used in topical creams and ointments to prevent infections in minor cuts, scrapes, or burns. The quantity of neomycin powder is included in the present invention in order to combat bacterial infections associated with later stages of bedsore. In more severe cases, bacterial infections can lead to sepsis with symptoms such as fever, chills, confusion, and hypotension. The quantity of neomycin powder severely hinders the growth of a broad set of gram-negative bacteria and some gram-positive bacteria, thus reducing the chances of bacterial infection in bedsores. In the preferred embodiment, the quantity of neomycin powder is about 1.25%, by weight, of the topical mixture.

Doxycycline is an antibiotic of the tetracycline class and is effective against both bacteria and protozoa. More specifically, doxycycline inhibits bacterial protein synthesis by binding to the 30S ribosomal subunit. Some of the main uses of doxycycline include bacterial pneumonia, acne, chlamydia infections, early Lyme disease, cholera, and syphilis. In particular, doxycycline is affective against Acinetobacter species, Bartonella bacilliformis, Brucella species, Klebsiella species, Klebsiella granulomatis, Campylobacter fetus, Enterobacter aerogenes, Escherichia coli, Francisella tularensis, Haemophilus ducreyi, Haemophilus influenza, Neisseria gonorrhoeae, Shigella species, Vibrio cholera, and Yersinia pestis. In the preferred embodiment, the quantity of doxycycline powder is about 2%, by weight, of the topical mixture.

The quantity of antifungal powder is another important ingredient necessary for adequate/proper healing of bedsores. Fungi reproduce through spores in the air and as a result can easily come into contact with skin. Patients with bedsores most often time have a weakened immune system as well as open wounds on the skin. These two conditions increase the chances of fungal infections in and/or around the open wounds, further diminishing the health of the patient. In order to prevent fungal infections, the present invention includes the quantity of antifungal powder. A variety of antifungals medication may be used for the quantity of antifungal powder constituent. In the preferred embodiment, a quantity of miconazole powder is used as the quantity of antifungal powder. In particular, the quantity of miconazole powder is about 2%, by weight, of the topical mixture.

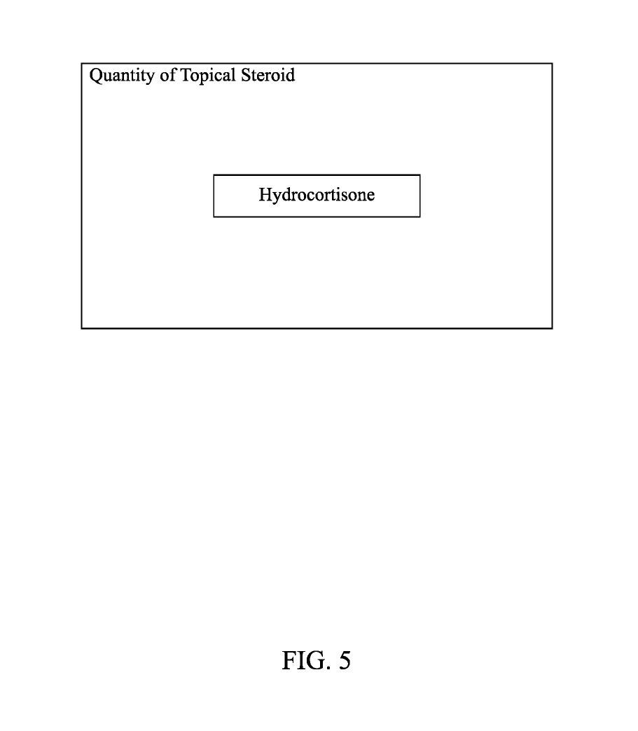

Referring to FIG. 5, The quantity of topical steroid is used to address the physical discomfort and pain associated with bedsores. More specifically, the preferred quantity of topical steroid powder is a quantity of hydrocortisone powder. Hydrocortisone is traditionally used to treat various skin conditions such as eczema, dermatitis, and rashes. In relation to bedsores, the quantity of hydrocortisone powder is included in order to order to reduce inflammation, pain, itching, and swelling in and around the bedsore(s), thus increasing the comfort level of the patient/individual during the healing process. The quantity of hydrocortisone powder achieves this by increasing the amount of corticosteroid in the body. Corticosteroids are naturally produced by the body and help the body remain healthy and well. In the preferred embodiment, the quantity of hydrocortisone powder is about 2%, by weight, of the topical mixture.

Ethoxydiglycol is an industrial solvent that is often used for skin care products in order to thin out products and decrease viscosity. The quantity of ethoxydiglycol is used in the present invention in order to create a paste formulation to allow for easy skin application. More specifically, the quantity of ethoxydiglycol is mixed with the quantity of gentamycin powder, the quantity of miconazole powder, the quantity of hydrocortisone powder, the quantity of neomycin powder, and the quantity of doxycycline powder to form a paste/cream. The quantity of ethoxydiglycol is dependent on the desired thickness of the paste. In the preferred embodiment, the quantity of ethoxydiglycol is about 40.63%, by weight, of the topical mixture.

In order to adequately inhibit bacterial growth in and/or around the bedsore, one of the constituents of the present invention an ingredient that directly destroys bacteria, the quantity of silver sulfadiazine cream. Silver sulfadiazine is a broad antimicrobial that is traditionally used to treat and prevent infections associated with burns as the silver sulfadiazine kills a wide array of bacteria. More specifically, silver sulfadiazine is a bactericidal, meaning the compound destroys bacteria, for many gram-negative and gram-positive bacteria as well as being effective against yeast. The quantity of silver sulfadiazine cream is about 50%, by weight, of the topical mixture.

The quantity of retinoic acid powder is used to aid the skin in the healing processes. Traditionally, retinoic acid is used in the treatment of photo aging and as an acne treatment. In relation to bedsores, the retinoic acid is used to refurbish the skin affected by bedsores by removing scabs and acne from said skin. In the preferred embodiment of the present invention, the quantity of retinoic acid powder is about 0.02%, by weight, of the topical mixture.

The quantity of dimethyl sulfoxide solution is used as a topical analgesic and to aid in the absorption of the various ingredients of the present invention by the skin. Dimethyl sulfoxide increases the rate of absorption of various compounds through the skin, thus enabling said compounds to penetrate various layers of the skin easier in order to produce a quicker response and facilitate the healing processes. In particular, dimethyl sulfoxide solution is often used to antifungal medications. In relation to bedsores, the quantity of dimethyl sulfoxide solution enables the constituents of the present invention, the antifungal powder and the antibiotic powder composition, to penetrate the skin in and around the wound. In the preferred embodiment, the quantity of dimethyl sulfoxide solution is about 0.10%, by weight, of the topical mixture.

The oleo plasticized base is used as the base for the topical mixture. The oleo plasticized base also adds a coating to the skin in order to prevent urine and human excrement from entering the wound. This is especially important as one region of the human body that is highly susceptible to bedsores is the back of the legs and the buttocks. In the preferred embodiment, the quantity of oleo plasticized base is added quantum satis in order to yield the topical mixture.

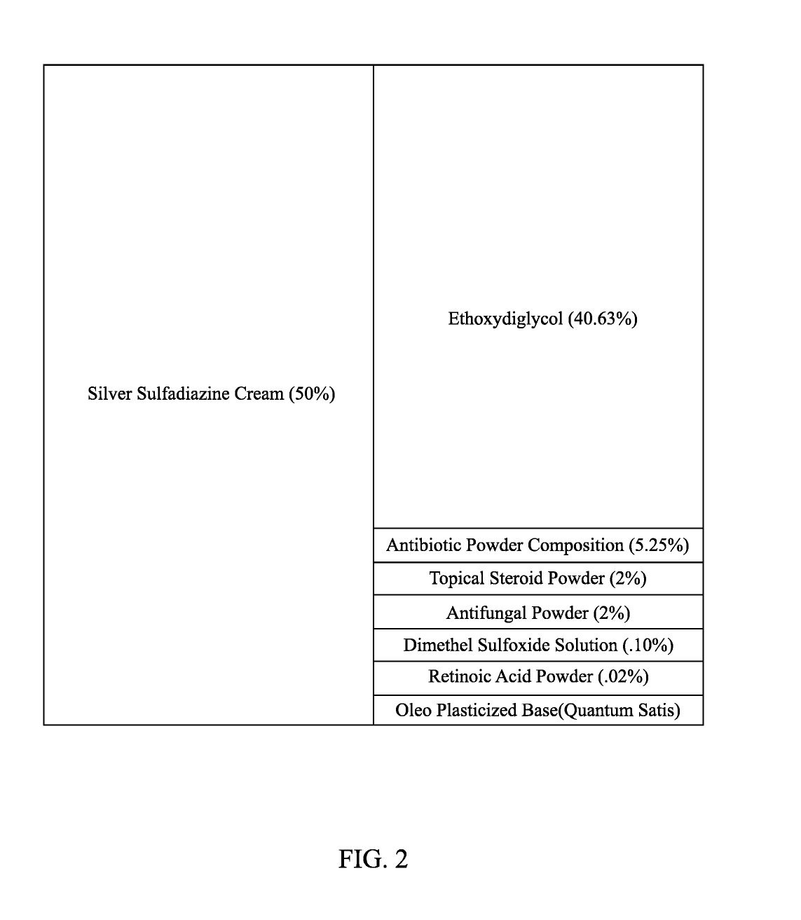

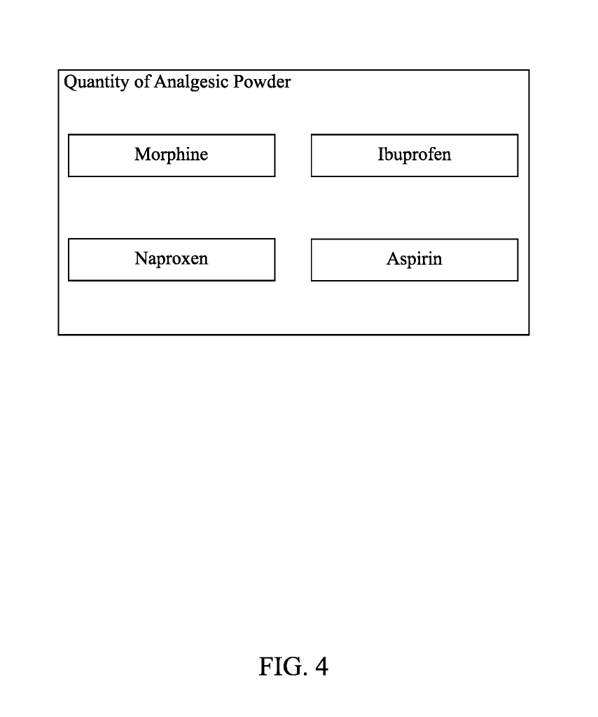

In one embodiment of the present invention, a quantity of topical analgesic powder is included in order to provide the patient/individual with additional pain relief. Bedsores can cause excruciating pain to the patient/individual, especially towards the later stages of the condition. The topical analgesic may be included in the present invention in order to decrease pain produced by the bedsores; the type of topical analgesic and quantity of said topical analgesic are subject to doctor's discretion. For example, referring to FIG. 4, the topical analgesic powder may be selected from a group consisting of: a quantity of morphine, a quantity of ibuprofen, a quantity of naproxen, a quantity of aspirin, and any combination thereof. Similar the preferred constituents of the present invention, the quantity of topical analgesic powder is heterogeneously combined into the topical mixture in order to be applied directly onto the bedsore(s). In the preferred embodiment, the present invention comprises 2 grams of gentamycin powder, 2 grams of miconazole powder, 2 grams of hydrocortisone, 1.25 grams of neomycin powder, 2 grams of doxycycline powder, 40.63 grams of ethoxydiglycol, 50 grams of silver sulfadiazine cream, 0.02 grams of retinoic acid powder, and 0.10 grams of dimethyl sulfoxide solution. Additionally, oleo plasticized base is added quantum satis. A percentage composition of each constituent is included in FIG. 2. More specifically, in the preferred embodiment, the present invention is formed by mixing the quantity of gentamycin powder, the quantity of miconazole powder, the quantity of hydrocortisone powder, the quantity of neomycin powder, and the quantity of doxycycline powder with the quantity of ethoxydiglycol to form a paste. The resulting paste is then mixed with the quantity of retinoic acid powder for one minute. Next, the quantity of silver sulfadiazine cream is added and the resulting mixture is mixed for two minutes. Next, the quantity of oleo plasticized base is added quantum satis to reduce the mixture to a volume of 100 grams. The final mixture is mixed for four minutes at a high speed within a high-sheer mixer in order to yield the final topical mixture. All levigations and mixing is performed by a high-sheer mixer. Alternative means for producing the present invention may be utilized as well.

The recommended usage of the present invention includes application of the topical mixture to the bedsore(s) twice a day. However, application of the present invention may be as needed or recommended by a medical professional. The present invention may be applied in conjunction with introvenous therapy, oral antibiotics, medicated bandages, or wound care gels, ointments, or creams.

Although the invention has been explained in relation to its preferred embodiment, it is to be understood that many other possible modifications and variations can be made without departing from the spirit and scope of the invention as hereinafter claimed.

What is claimed is:

1. A decubitus ulcer salve comprises:
a quantity of antibiotic powder composition;
a quantity of antifungal powder;
a quantity of topical steroid powder;
a quantity of ethoxydiglycol;
a quantity of silver sulfadiazine cream;
a quantity of retinoic acid powder;
a quantity of dimethyl sulfoxide solution;
a quantity of oleo plasticized base; and
the quantity of antibiotic powder composition, the quantity of antifungal powder, the quantity of topical steroid powder, the quantity of ethoxydiglycol, the quantity of silver sulfadiazine cream, the quantity of retinoic acid powder, the quantity of dimethyl sulfoxide solution, and the quantity of oleo plasticized base being heterogeneously combined into a topical mixture.

2. The decubitus ulcer salve as claimed in claim 1 comprises:
the quantity of the antibiotic powder composition being about 5.25% by weight (wt %) of the topical mixture;
the quantity of the antifungal powder being about 2% wt of the topical mixture;
the quantity of topical steroid powder being about 2% wt of the topical mixture;
the quantity of ethoxydiglycol being about 40.63% wt of the topical mixture;
the quantity of silver sulfadiazine cream being about 50% wt of the topical mixture;
the quantity of retinoic acid powder being about 0.02% wt of the topical mixture;
a quantity of dimethyl sulfoxide solution being about 0.10% wt of the topical mixture; and
the quantity of oleo plasticized base being about quantum satis of the topical mixture.

3. The decubitus ulcer salve as claimed in claim 2 comprises:
the quantity of antibiotic powder composition comprises a quantity of gentamycin powder, a quantity of neomycin powder, and a quantity of doxycycline powder;
the quantity of gentamycin powder being about 2% of the topical mixture;
the quantity of neomycin powder being about 1.25% of the topical mixture; and
the quantity of doxycycline powder being about 2% of the topical mixture.

4. The decubitus ulcer salve as claimed in claim 1, wherein the quantity of topical steroid is a quantity of hydrocortisone.

5. The decubitus ulcer salve as claimed in claim 1, wherein the antibiotic powder composition is selected from a group consisting of: a quantity of gentamycin powder, a quantity of neomycin powder, a quantity of doxycycline powder, and any combination thereof.

6. The decubitus ulcer salve as claimed in claim 1, wherein the oleo plasticized base is added quantum satis to form a cream/paste formulation.

7. The decubitus ulcer salve as claimed in claim 1 comprises:
a quantity of topical analgesic powder; and
the quantity of topical analgesic powder being heterogeneously combined into the topical mixture.

8. The decubitus ulcer salve as claimed in claim 7, wherein the topical analgesic powder is selected from a group consisting of: a quantity of morphine, a quantity of ibuprofen, a quantity of naproxen, a quantity of aspirin, and any combination thereof.

9. A decubitus ulcer salve comprises:
a quantity of antibiotic powder composition;
a quantity of antifungal powder;
a quantity of topical steroid powder;
a quantity of ethoxydiglycol;
a quantity of silver sulfadiazine cream;
a quantity of retinoic acid powder;
a quantity of dimethyl sulfoxide solution;
a quantity of oleo plasticized base;
the quantity of antibiotic powder composition, the quantity of antifungal powder, the quantity of topical steroid powder, the quantity of ethoxydiglycol, the quantity of silver sulfadiazine cream, the quantity of retinoic acid powder, the quantity of dimethyl sulfoxide solution, and the quantity of oleo plasticized base being heterogeneously combined into a topical mixture;
the quantity of the antibiotic powder composition being about 5.25% by weight (wt %) of the topical mixture;
the quantity of the antifungal powder being about 2% wt of the topical mixture;
the quantity of topical steroid powder being about 2% wt of the topical mixture;
the quantity of ethoxydiglycol being about 40.63% wt of the topical mixture;
the quantity of silver sulfadiazine cream being about 50% wt of the topical mixture;
the quantity of retinoic acid powder being about 0.02% wt of the topical mixture;
a quantity of dimethyl sulfoxide solution being about 0.10% wt of the topical mixture; and
the quantity of oleo plasticized base being about quantum satis of the topical mixture.

10. The decubitus ulcer salve as claimed in claim 9 comprises:
the quantity of antibiotic powder composition comprises a quantity of gentamycin powder, a quantity of neomycin powder, and a quantity of doxycycline powder;
the quantity of gentamycin powder being about 2% of the topical mixture;
the quantity of neomycin powder being about 1.25% of the topical mixture; and
the quantity of doxycycline powder being about 2% of the topical mixture.

11. The decubitus ulcer salve as claimed in claim 9, wherein the quantity of topical steroid is a quantity of hydrocortisone.

12. The decubitus ulcer salve as claimed in claim 9, wherein the antibiotic powder composition is selected from a group consisting of: a quantity of gentamycin powder, a quantity of neomycin powder, a quantity of doxycycline powder, and any combination thereof.

13. The decubitus ulcer salve as claimed in claim 9, wherein the oleo plasticized base is added quantum satis to form a cream/paste formulation.

14. The decubitus ulcer salve as claimed in claim 9 comprises:
a quantity of topical analgesic powder; and
the quantity of topical analgesic powder being heterogeneously combined into the topical mixture.

15. The decubitus ulcer salve as claimed in claim 14, wherein the topical analgesic powder is selected from a group consisting of: a quantity of morphine, a quantity of ibuprofen, a quantity of naproxen, a quantity of aspirin, and any combination thereof.
